(19) United States
(12) Plant Patent Application Publication
(10) Pub. No.: US 2003/0167547 P1
(43) Pub. Date: Sep. 4, 2003

(54) Apple variety named 'CJ07'

(76) Inventor: Peter Collinge, Napier (NZ)

Correspondence Address: Michelle Bos, Stratton Ballew PLLC, 213 South 12th avenue, Yakima, WA, 98902, US

(21) Appl. No.: 10/032,692

(22) Filed: Dec. 27, 2001

(51) Int. Cl. A01H 5/00

(52) U.S. Cl. PLT/161

(57) ABSTRACT

A new and distinct apple variety is described. The variety results from a whole tree mutation of the variety Sciearly (PP11,092). The fruit of the apple tree of this new variety has an attractive appearance characterised by intense red block colour, early harvest maturity, and large fruit size relative to other early season cultivars. The new variety is known by the temporary denomination CJ07.

### SUMMARY OF THE INVENTION

The present invention relates to a new and distinct apple variety. The new variety, with the temporary denomination CJ07, was discovered in January 1996 as a whole tree mutation of ‘Sciearly’ (P11,092). The sport arose on a tree planted in the discoverer's orchard in Hawkes Bay, New Zealand. The block of trees of Sciearly was propagated on MM106 rootstock and had been established on the orchard as part of the original release of the Sciearly variety in New Zealand.

The mutation was originally noted due to its early development of bright red fruit colour. All fruit on the tree appeared to be consistently ahead in colour development than fruit on surrounding Sciearly trees obtained from the same original source. The colour development was particularly notable considering the parent variety was selected for its early harvest season and dark red block colour.

Budwood was taken from the original tree at the time of discovery and 140 trees were propagated on MM106 rootstock, and 100 trees on M793 rootstock. These trees were planted on the discoverer's orchard property in winter 1997 and produced their first crop in January 1999. The colour and harvest maturity period of fruit on these trees was found to be consistent with the original tree, and true to type.

A further generation of trees were produced and were established in the discoverer's orchard in winter 1998. Fruit from these trees have exhibited the early fruit colour development and typical red colour of the original mutation over three seasons.

The new variety was determined to be distinct in comparison with the parent variety Sciearly by the following characteristics:

fruit colour at harvest maturity which is a brighter, more intense red.

an earlier harvest season by at least a week, determined on the basis of background colour development.

### BRIEF DESCRIPTION OF PHOTOGRAPHS

The accompanying photographs show typical specimens of the young tree, flowers, foliage and fruit of the new variety as depicted in colours as nearly true as is reasonably possible to make the same in a colour illustration of this character.

FIG. 1 shows a typical fruit of the apple variety CJ07 photographed in situ on the original tree of the whole tree mutation.

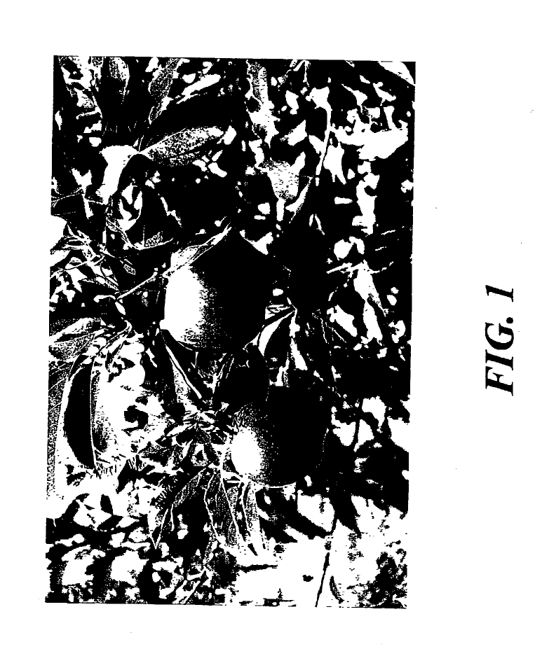

FIG. 2 shows stem end and side views, showing both blush and shade sides, of a typical fruit of the apple variety CJ07. The fruit shown in FIG. 2 was obtained from trees asexually propagated from the original whole tree mutation.

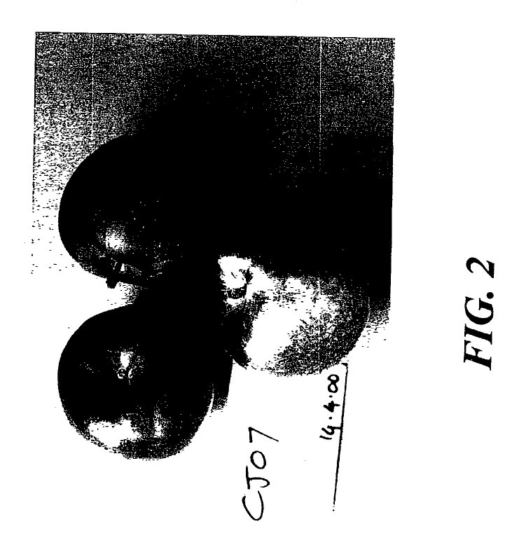

FIG. 3 shows a young tree (3^{rd} leaf in the orchard) showing foliage and typical branching development.

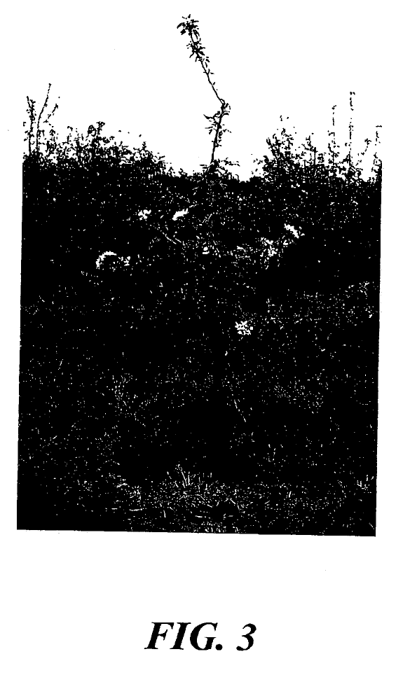

FIG. 4 shows typical flower and foliage of the variety CJ07.

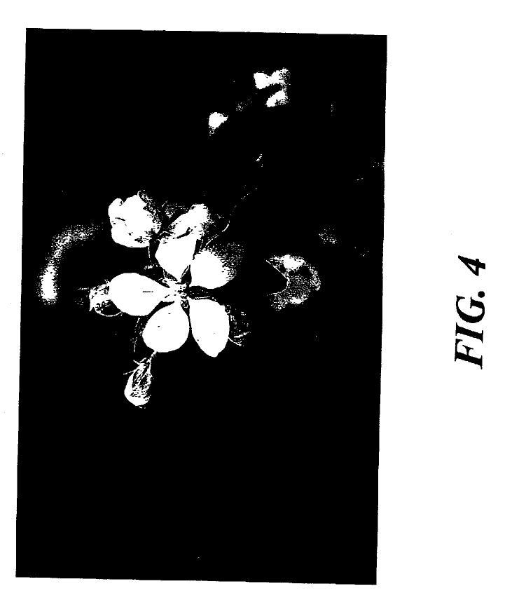

### MORPHOLOGICAL DESCRIPTION OF THE VARIETY

The following is a detailed description of the new variety with colour terminology in accordance with The Royal Horticultural Society Colour Chart (RHSCC) 2001 edition.

The specimens described were grown at the discoverer's property at 1999 Pakowhai Road, RD 3, Napier, New Zealand. The observations were made in the 2000-2001 season on trees on MM. 106 rootstock that were four years old at the time, and managed under standard orchard practice. It should be understood that the characteristics described will vary depending upon cultural practices, climatic conditions, planting location and season.

Tree: Medium size; spreading habit; bearing on both one-year wood and spurs; medium vigour on MM. 106 rootstock.

Trunk: Moderately rough; circumference 240 mm at a height of 200 mm; the bark is grey-green RHSCC 197C when mature.

Branches: Bark moderately rough, grey-green RHSCC 197C when mature; moderate branching, in a whorl arrangement, the angle of branching being typically 20 degrees above the horizontal; approximately 5 spurs per 100 mm of growth on two year old wood; length of annual growth 300-350 mm (measured on extension shoots arising from branches arising approximately 1.5 m above ground level).

Lenticels: Medium size; average numbers.

Leaves: Average length 106 mm; average width 53 mm; upward pose; upfolded to concave shape in cross section; serrate indentation of margin; medium glossiness on upper surface; weak pubescence on lower side; petiole length 30-45 mm; small to insignificant stipule size; colour of blade is green RHSCC 137A.

Flowers: Medium blooming period, starting about 5 October for 2 weeks; medium size; five petals, margin of petals not touching; bud colour pink RHSCC 55b fading to white as the flower opens; five sepals small size, pointed, green with red tips; pubescent ovary.

Fruit: Examined at optimum. The main harvest period typically is from about 11 February until 25 February in Hawkes Bay, New Zealand.

Size: Large to very large; average width, 76 mm; average height, 62 mm; average weight, 200 g.

Shape: Short globose, conical; symmetrical in side view; ribbing absent; medium degree of crowning at distal end.

Cavity: Average width, 36 mm.; average depth, 12 mm.

Basin: Average width, 28 mm; average depth, 90 mm.

Stem: Medium thickness; medium length, 24 mm.

Sepal: Medium length; spacing, touching.

Eye: Size, medium; aperture, closed.

Skin: Smooth; bloom of skin, absent; greasiness of skin, slight; cracking tendency of skin, absent; thickness, medium; background colour, yellow-green RHSCC 145C; lenticels, small.

Over Colour: Approximately 80% of skin surface; bright red, RHSCC 46B; some russet around stem end cavity.

Flesh: Medium firm, juicy and crisp; tinged light yellow RHSCC 10D.

Texture: Medium.

Flavour: Sweet; medium acidity; mild aroma.

Quality: Excellent.

Sinus: Closed.

Seeds: Five locules; 1 to 2 seeds per locule; 8-10 seeds total; medium size, average 10 mm long and 5 mm wide; colour, brown RHSCC 175A.

Use: Dessert.

Keeping quality: Excellent.

Production: Early season, regular cropping, although slight tendency to biennial variation in production volume.

Management: Thinning of the crop load in early summer is recommended and tailoring pruning to account for the slight biennial tendency.

1. We claim a new and distinct apple variety substantially as described in the specification and illustrations above, arising as a whole tree mutation of the apple variety known as ‘Sciearly’ (U.S. Plant Pat. No. 11,092) and characterised by its brighter, more intense red fruit colour at harvest maturity and an earlier harvest season by at least a week, determined on the basis of background colour development.
